(19) United States
(12) Patent Application Publication
(10) Pub. No.: US 2008/0262479 A1
(43) Pub. Date: Oct. 23, 2008

(54) MEDICAL DEVICE FOR PREVENTING PASSIVE REGURGITATION

(76) Inventor: DANIEL BARELA, Albuquerque, NM (US)

Correspondence Address: KENNEDY, LTD., 3916 JUAN TABO NE, SUITE 14, ALBUQUERQUE, NM, 87110, US

(21) Appl. No.: 11/738,303

(22) Filed: Apr. 20, 2007

(51) Int. Cl. A61B 17/00

(52) U.S. Cl. 606/1

(57) ABSTRACT

The present invention includes a medical device including a base member having a receiving element and an engagement member connected to the base member. In the various embodiment described below, the engagement member includes a user end, an engagement end, and a locking mechanism. The locking mechanism is adapted to interface with the receiving element in response to a user input such that the engagement end engages a tracheal region of a patient thereby substantially prohibiting flow through the esophagus of the patient. In other embodiments of the present invention, the medical device further includes a retaining strap adapted to secure the base member about a neck region of the patient, thus prohibiting movement of the base member relative to the tracheal region of the patient.

### BACKGROUND

1. Field of the Invention

The present invention relates generally to the field of medical devices, and more particularly for medical devices usable in the provision of emergency and surgical medical services.

2. History of the Related Art

In the United States alone, cardiac arrests, pulmonary arrests and drug overdoses occur over four million times per year. The bulk of these incidences occur outside of the hospital setting, and thus to the extent that a patient receives immediate medical care, it is often at the hands of a trained first responder, such as an emergency medical technician, firefighter, police officer or military personnel. Given the aging population and continuing deterioration of the health of the general public, this number of incidences is expected to increase in the future, thus placing an even greater strain on the corps of first responders.

Although each of these types of incidences have distinct causes, they do share an unfortunate side effect that often results in serious complications, including death. During any one of these types of attacks, it is often the case that the patient's gastro-esophageal sphincter will relax, permitting the flow of the patient's stomach contents out of the stomach towards the patient's mouth. This side effect is known in the medical field as passive regurgitation or gastric reflux. In countless cases, the unconscious patient unwittingly will aspirate his own stomach contents into his lungs, resulting in death by drowning. In those cases that do not result in death, numerous complications ranging from choking to severe brain damage can arise, dramatically increasing the cost of care for that patient.

Currently, first responders and other medical professionals such as anesthesiologists and surgeons are trained to minimize the potential for passive regurgitation by manually applying pressure to the cricoid cartilage that surrounds the trachea of a patient. The pressure applied to the cricoid compresses the esophagus of the patient, thus preventing any of the gastric reflux from entering the patient's trachea and/or lungs. While this manual procedure can be employed readily in hospital settings without distracting from patient care, employment of the manual procedure in emergency medical situations presents a number of problems. First, the manual application of cricoid pressure requires at least one hand from a first responder that would otherwise be used for performing other critical life saving measures. Second, the pressure must be applied constantly and consistently for long periods up to thirty minutes, and fatigue, distraction, or other emergencies can cause the first responder to lose constancy or consistency in the performance of the task. Third, the manual application of the procedure might interfere with other necessary medical tasks, such as restarting a patient's heart using a defibrillator. Fourth, it can be the case that there is only a single first responder who must handle all of the patient care on his or her own. In these circumstances, the first responder will often forego the application of cricoid pressure in order to tend to more urgent life-saving measures.

To be sure, the manual application of cricoid pressure is well suited for some circumstances. However, there remains a need in the art for a device, system or method of applying cricoid pressure that does not have any of the shortcomings of the manual procedure noted above. In particular, there is a need in the art for a device, system or method of applying cricoid pressure that is relatively automated, constant, consistent, and can be performed by a single medical professional.

### SUMMARY OF THE PRESENT INVENTION

Accordingly, the present invention includes a medical device including a base member having a receiving element and an engagement member connected to the base member. In the various embodiment described below, the engagement member includes a user end, an engagement end, and a locking mechanism. The locking mechanism is adapted to interface with the receiving element in response to a user input such that the engagement end engages a tracheal region of a patient thereby substantially prohibiting flow through the esophagus of the patient. In other embodiments of the present invention, the medical device further includes a retaining strap adapted to secure the base member about a neck region of the patient, thus prohibiting movement of the base member relative to the tracheal region of the patient. Additionally, the medical device can further include a cushion disposed on the engagement end of the engagement member. The cushion can be adapted to interface with the tracheal region of the patient, and in particular with the cricoid cartilage of the patient during use of the medical device. These and other features, aspects and advantages of the present invention are illustrated more clearly below in a description of the preferred embodiments, which is made with reference to the following figures.

### BRIEF DESCRIPTION OF THE FIGURES

FIG. 1 is a perspective view of a medical device in accordance with a preferred embodiment of the present invention.

FIG. 2 is a front view of the medical device shown in FIG. 1.

FIG. 3 is a top view of the medical device shown in FIG. 1.

FIG. 4 is a bottom view of the medical device shown in FIG. 1.

FIG. 5 is a cross-sectional view of the medical device shown in FIG. 1.

FIG. 6 is a cross-sectional view of a portion of a medical device in accordance with an alternative embodiment of the present invention.

FIG. 7 is a cross-sectional view of a portion of a medical device in accordance with the alternative embodiment shown in FIG. 6.

FIG. 8 is a schematic illustration of a medical device in use in accordance with the preferred embodiment of the present invention.

FIG. 9 is a schematic illustration of the medical device in use in accordance with the preferred embodiment shown in FIG. 8.

### DETAILED DESCRIPTION OF THE PREFERRED EMBODIMENTS

The following description of the preferred embodiment of the invention is not intended to limit the invention to this preferred embodiment, but rather to enable any person skilled in the art of medical devices to make and use this invention. In particular, the figures and preferred embodiment described herein should be construed as merely illustrative in nature, and are in no way intended to limit the scope of the present invention as defined in the following claims.

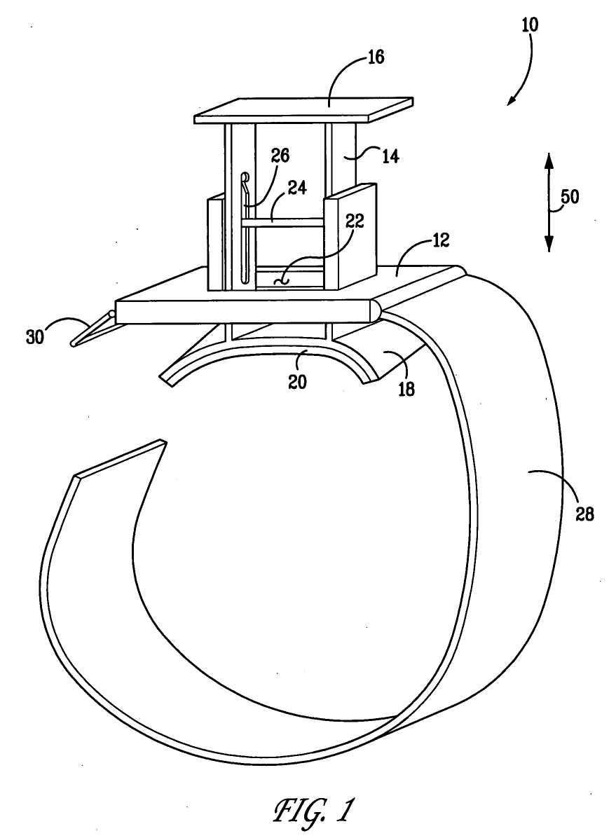

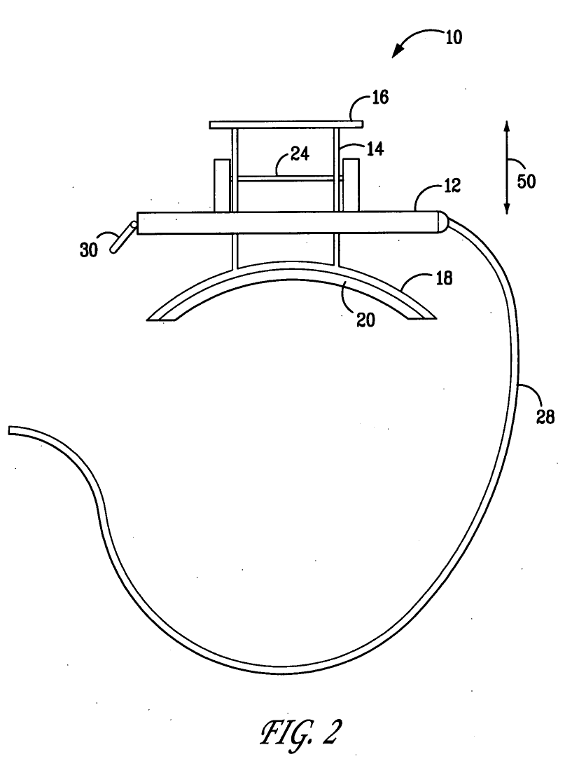

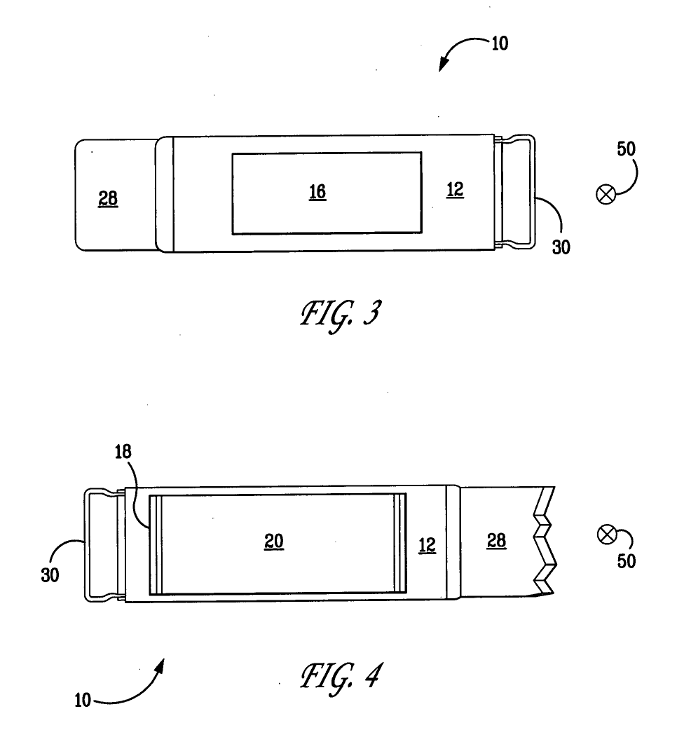

As shown in FIGS. 1 through 4, a medical device 10 according to a preferred embodiment includes a base member 12 having a receiving element 24 attached thereto. The medical device 10 further includes an engagement member 14 connected to the base member 12 and defining a user end 16, an engagement end 18, and a locking mechanism 26. In the medical device of the preferred embodiment, the locking mechanism 26 is adapted to interface with the receiving element 24 in response to a user input such that the engagement end engages a tracheal region of a patient in order to substantially prohibit flow through the esophagus of the patient. In particular, the medical device 10 of the preferred embodiment is well-suited for engaging the cricoid of a patient, and subsequent to user input, applying pressure to the cricoid thereby substantially collapsing the esophagus of the patient and substantially prohibiting passive regurgitation.

The base member 12 of the medical device 10 of the preferred embodiment can be composed of any suitable material for medical applications, such as for example any combination of a monomer, polymer, elastomer, alloy or metal that can be sterilized and/or re-sterilized after use. Alternatively, the base member 12 can be composed of a material that is subject to sterilization once, and is disposable after use. The base member 12 functions to stabilize the medical device 10 and to direct and constrain the motion of the engagement member 14 relative to the tracheal region of the patient. The base member 12 can define any suitable shape or geometry for performing its desired function, i.e. it can be substantially rectangular in geometry or it can be curved or contoured to approximate the shape of a patient's neck. The base member 12 can further include other means or mechanisms to ease handling by a user, which are omitted from the figures for the sake of clarity.

The engagement member 14 of the medical device 10 of the preferred embodiment can also be composed of any suitable material for medical applications, such as for example any combination of a monomer, polymer, elastomer, alloy or metal that can be sterilized and/or resterilized after use. Alternatively, the engagement member 14 can be composed of a material that is subject to sterilization once, and is disposable after use. The engagement member 14 functions to transfer a constant and consistent pressure to a tracheal region of a patient in order to substantially prohibit passive regurgitation. The engagement member 14 can define any suitable shape or geometry for performing its desired function, i.e. it can be substantially rectangular in geometry or it can be curved or contoured to approximate the shape of a patient's neck. For example, the engagement end 18 of the engagement member 14 shown in FIGS. 1 through 4 is curved in order to more efficiently transfer pressure to the cricoid of a patient. The engagement member 14 can further include other means or mechanisms to ease handling by a user, which are omitted from the figures for the sake of clarity.

In a first variation to the medical device 10 of the preferred embodiment, the medical device 10 can further include a retaining strap 28 connected to the base member 12. The retaining strap 28 functions to secure the base member 12 about the neck region of the patient, and in particular about the tracheal region of the patient. The retaining strap 28 can be composed of any suitable material including for example a textile material, rubber or rubberized materials, a polymer, elastomer or any other type of synthetic material. As shown in FIGS. 1 through 4, a variation of the medical device 10 can further include a hook or loop 30 adapted to receive and immobilize the retaining strap 28. In one example variation, the hook 30 can include a prong (not shown) for selectively engaging one or more holes (not shown) in the retaining strap 28. In another example variation, the hook 30 can function as a passage through which the retaining strap 28 can pass and thereafter couple to itself through various means known in the art, such as for example a Velcro® closure.

In a second variation of the medical device 10 of the preferred embodiment, the medical device 10 can further include a cushion 20 disposed on the engagement end 18 of the engagement member 14. The cushion 20 functions to interface with the tracheal region of the patient and to constantly and consistently distribute pressure to that region, and in particular to the patient's cricoid. Some suitable materials include for example polyurethane foam or composite or synthetic materials that have a density and sterilizable character appropriate for interfacing directly with a patient.

In a third variation of the medical device 10 of the preferred embodiment, the base member 12 further includes a passage 22 through which the engagement member 14 may pass in a longitudinal direction. As used herein, the term longitudinal direction includes any direction that is substantially normal to a top surface defined by the base member 12, i.e. the direction in which pressure is applied to the cricoid in order to substantially prohibit passive regurgitation. For purposes of illustration, the longitudinal direction is approximated in the figures as being substantially parallel to the arrow 50. The passage 22 functions to contain the engagement member 14, and to direct and constrain the motion of the engagement member 14 prior to and during the application of pressure to the cricoid by the engagement member 14. The passage 22 can fully enclose the engagement member 14 or it can partially enclose the engagement member 14.

In a fourth variation of the medical device 10 preferred embodiment, the retaining element 24 can include a pin disposed substantially perpendicular to the longitudinal direction in which the engagement member moves, i.e. substantially perpendicular to arrow 50. The pin 24 can be composed of suitable material for medical applications, such as for example any combination of a monomer, polymer, elastomer, alloy or metal that can be sterilized and/or re-sterilized after use. Alternatively, the pin 24 can be composed of a material that is subject to sterilization once, and is disposable after use. The pin 24 can be of any geometry, including the substantially cylindrical geometry shown in FIGS. 5 through 9. Alternatively, the pin 24 can have substantially elliptical or polygonal cross-sectional geometries for engaging with the locking mechanism 26.

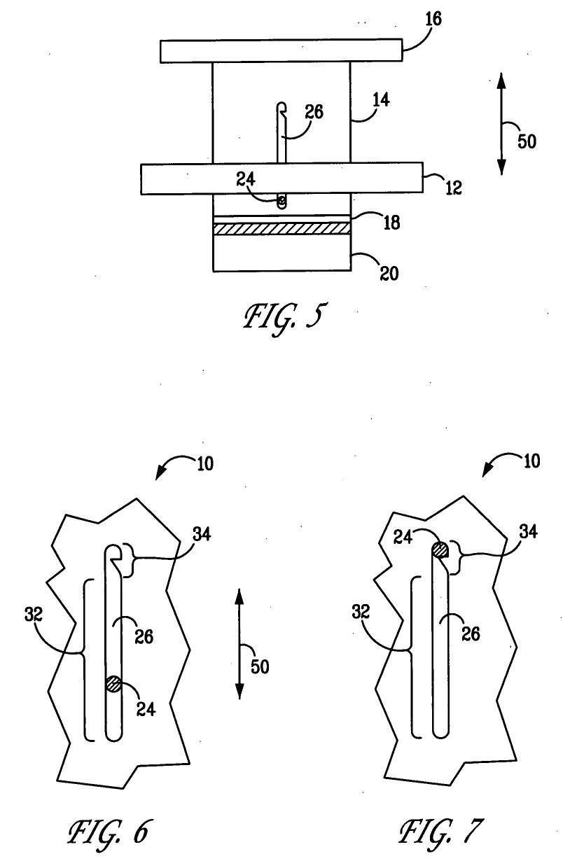

In a fifth variation of the medical device 10 of the preferred embodiment, the locking mechanism 26 includes a channel. As shown in FIGS. 6 and 7, the channel 26 can include a travel region 32 and a locking region 34 that are connected to each other and function to permit the receiving element 24 to selectively move therein. In particular, as shown in FIG. 6, the receiving element 24 can move in the direction of arrow 50 provided that it remains in the travel region 32 of the channel 26. However, as shown in FIG. 7, once the receiving element 24 is moved into the locking region 34, it is effectively and substantially prohibited from any further motion in the direction of arrow 50. As such, the channel 26 functions to selectively permit that motion of the receiving element 24, and by extension, the channel 26 functions to selectively permit the motion of the engagement member 14 relative to the base member 12.

In one alternative to the fifth variation, the channel 26 defines a predefined length in the longitudinal direction, i.e. substantially parallel to arrow 50. The predefined length can be selected in order to determine an optimal force (Newtons) and/or pressure (Newtons/meter) applied to the cricoid by the engagement member 14 to ensure that the esophagus of the patient is sufficiently constricted. Research has shown that the optimal force to be applied to the cricoid in order to inhibit passive regurgitation is within a range of twenty to forty Newtons. Accordingly, the optimal pressure to be applied to the cricoid can be varied and/or controlled by determining the area and/or mechanical deformation properties of the engagement end 18 of the engagement member, which can include the cushion 20 noted above.

Alternatively, the predefined length can be selected in order to correspond to an optimal stroke length of the engagement member 14, wherein the optimal stroke length is defined as the distance that the engagement member 14 must travel in the longitudinal direction in order to sufficiently constrict the esophagus of the patient. Research has shown that the optimal stroke length falls within a range between one and two inches for the manual application of pressure to the cricoid.

In determining the optimal force, pressure and/or stroke length associated with the channel 26, the retaining strap 28 described above can further include one or more mechanisms or means for ensuring that the force, pressure and/or stroke length is optimized. As such, the retaining strap 28 can be composed of materials that exhibit certain desirable mechanical properties with respect to tensile strength, deformation (i.e. stretching) and torsion strength. Alternatively, the retaining strap 28 can include one or more markers or indicators for advising a user as to the appropriate tension needed to secure the base member 12 about the patient's neck region in order to ensure the appropriate force, pressure and/or stroke length applied by the engagement member 14.

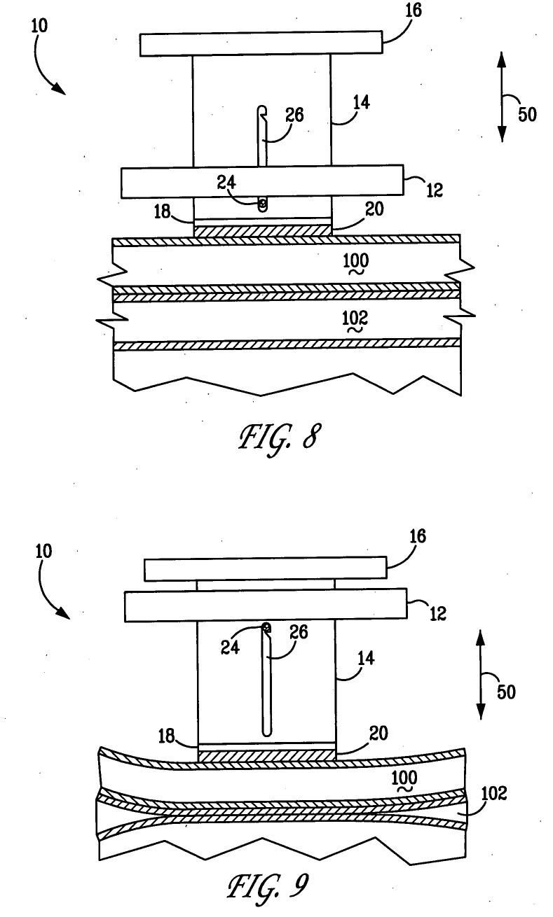

Operation of the medical device 10 of the preferred embodiment is schematically illustrated in FIGS. 8 and 9. As shown in FIG. 8, the medical device 10 of the preferred embodiment is disposed by a user in the tracheal region of a patient, which includes a trachea 100 disposed nearest to the medical device 10 and an esophagus 102 disposed behind or to the interior of the trachea 100. As noted above, a suitable means for substantially prohibiting passive regurgitation is the application of pressure to the cricoid cartilage (not shown), which forms part of the trachea 100.

In operation, a user positions the medical device 10 of the present invention in the tracheal region and secures the base portion 12 about the patient's neck using for example the retaining strap (not shown) described above or some other suitable means or mechanism for securing the base member 12 in the optimal position. Prior to use, the locking mechanism 26 has not engaged the receiving element 24 and the medical device 10 is in a traveling position. As such the amount of force or pressure applied to the cricoid is limited to the contact force from the engagement member 18 and/or cushion 20.

In order to apply pressure to the cricoid, the user applies a force to the user end 16 of the engagement member 14 in the longitudinal direction, which as noted before is substantially parallel to arrow 50. As the base member 12 is substantially secured and motionless relative to the trachea 100, the engagement member 14 travels in the longitudinal direction applying the force against the cricoid. Since the cricoid cartilage is at least semi-rigid, it will only flex and/or deform a limited amount. The remainder of the force applied by the user that is not used in flexing and/or deforming the cricoid will be translated directly to the esophagus 102. Because the esophagus 102 is not rigid or semi-rigid, it will be readily deformed and constricted as shown in FIG. 9, thereby substantially prohibiting any passive regurgitation.

As noted above, one feature of the medical device 10 of the preferred embodiment is that the locking mechanism 26 can engage the receiving element 26 in such a manner that further motion of the engagement member 14 relative to the base member 12 is substantially negated. Accordingly, if a user enters the medical device 10 into a locked position as shown in FIG. 9, then no further action is required on the user's part. Unlike the current methods described above, the proper use of the medical device 10 of the preferred embodiment permits the user to attend to other activities without having to constantly and consistently apply pressure to the patient's cricoid, thereby allowing the user to tend to other life-saving measures.

As a person skilled in the art of medical devices will recognize from the previous detailed description and from the figures and claims, modifications and changes can be made to the preferred embodiment of the invention without departing from the scope of this invention defined in the following claims.

1. A medical device comprising:
a base member comprising a receiving element; and
an engagement member connected to the base member, the engagement member comprising a user end, an engagement end, and a locking mechanism;
wherein the locking mechanism is adapted to interface with the receiving element in response to a user input such that the engagement end engages a tracheal region of a patient thereby substantially prohibiting flow through the esophagus of the patient.

2. The medical device of claim 1 further comprising a retaining strap adapted to secure the base member about a neck region of the patient.

3. The medical device of claim 1 further comprising a cushion disposed on the engagement end of the engagement member, the cushion adapted to interface with the tracheal region of the patient.

4. The medical device of claim 1 wherein the base member further comprises a passage through which the engagement member may pass in a longitudinal direction.

5. The medical device of claim 1 wherein the receiving element comprises a pin disposed substantially perpendicular to a longitudinal direction in which the engagement member moves.

6. The medical device of claim 1 wherein the locking mechanism comprises a channel defining a travel region and a locking region, wherein the receiving element can move when disposed in the travel region and wherein the receiving element is substantially prohibited from movement when disposed in the locking region.

7. The medical device of claim 6 wherein the channel comprises a predefined length in the longitudinal direction.

8. The medical device of claim 7 wherein the predefined length substantially corresponds to a stroke length of the engagement member.

9. The medical device of claim 8 wherein the stroke length is between 1 and 2 inches.

10. The medical device of claim 9 wherein the stroke length is approximately 1.5 inches.

11. A medical device for engaging a tracheal region of a patient, the medical device comprising:
a base member comprising a passage and a receiving element;
an engagement member connected to the base member and adapted to selectively traverse through the passage in a longitudinal direction in response to a user input, the engagement member defining a user end, an engagement end and a locking mechanism disposed between the user end and the engagement end;
wherein the locking mechanism is adapted to selectively interface with the receiving element thereby substantially prohibiting the engagement member from further traverse through the passage.

12. The medical device of claim 11 further comprising a retaining strap adapted to secure the base member about a neck region of the patient.

13. The medical device of claim 11 further comprising a cushion disposed on the engagement end of the engagement member, the cushion adapted to interface with the tracheal region of the patient.

14. The medical device of claim 11 wherein the receiving element comprises a pin disposed substantially perpendicular to the longitudinal direction in which the engagement member moves.

15. The medical device of claim 11 wherein the locking mechanism comprises a channel defining a travel region and a locking region, wherein the receiving element can move when disposed in the travel region and wherein the receiving element is substantially prohibited from movement when disposed in the locking region.

16. The medical device of claim 15 wherein the channel comprises a predefined length in the longitudinal direction.

17. The medical device of claim 16 wherein the predefined length substantially corresponds to a stroke length of the engagement member.

18. The medical device of claim 17 wherein the stroke length is between 1 and 2 inches.

19. The medical device of claim 17 wherein the stroke length is approximately 1.5 inches.

20. A medical device for engaging a tracheal region of a patient, the medical device comprising:
a base member comprising means for receiving;
an engagement member connected to the base member, the engagement member defining a user end and an engagement end; and
means for locking the engagement member relative to the base member;
wherein the engagement end is adapted to engage a tracheal region of a patient thereby substantially prohibiting flow through the esophagus of the patient in response to a user input and wherein the means for receiving cooperates with the means for locking in order to substantially prohibit motion of the engagement member relative to the base member.
